(19) United States
(12) Patent Application Publication
(10) Pub. No.: US 2024/0252446 A1
(43) Pub. Date: Aug. 1, 2024

(54) DERIVATIZED COLLAGEN-HYALURONIC ACID BASED IMPLANTS FOR SUSTAINED DELIVERY OF OPHTHALMIC DRUGS

(71) Applicant: SUSTAINED DRUG DELIVERY LLC, LOUISVILLE, KY (US)

(72) Inventors: DALE P. DeVORE, CHELMSFORD, MA (US); RICHARD A. EIFERMAN, LOUISVILLE, KY (US)

(21) Appl. No.: 18/103,620

(22) Filed: Jan. 31, 2023

(51) Int. Cl. A61K 9/70; A61F 9/00; A61K 9/00; A61K 31/573; A61K 31/7036; A61K 47/42

(52) U.S. Cl. CPC A61K 9/7007; A61F 9/0017; A61K 9/0051; A61K 31/573; A61K 31/7036; A61K 47/42

(57) ABSTRACT

The present invention is directed to collagen-hyaluronic acid based constructs for sustained delivery of agents and drugs to ocular tissues. In certain configurations, the constructs are prepared by a method comprising combining a derivatized collagen with a hyaluronic acid. In other configurations, the constructs are prepared by a method comprising combining a hyaluronic acid-poly-L-lysine (PLL) polymer with the derivatized collagen.

### FIELD OF INVENTION

The present invention describes derivatized collagen-hyaluronic acid based constructs for sustained delivery of agents and drugs to ocular tissues. In certain configurations, the constructs are in the form of single layer films or membranes, multilayer films or membranes, unidirectional single layer films or membranes and unidirectional multilayer films or membranes. While the described constructs may be particularly suited for ocular drug delivery, the constructs are equally applicable to other fields of medicine and dentistry and are, for example, suitable for veterinary use.

### BACKGROUND

Glaucoma is a leading cause of blindness worldwide. While the exact etiology is unknown, there is either overproduction or impaired exit of aqueous humor from the eye causing elevated intraocular pressure (IOP). Elevated IOP causes damage to the optic nerve and, if untreated, can result in loss of vision. There are also many sub-types of glaucoma. The initial treatment for glaucoma is usually topical medication to lower the intraocular pressure by either reducing aqueous production or increasing aqueous outflow through the trabecular meshwork or uveal-scleral channels. Oral medication is also available, but rarely used on a long term basis due to side effects. Surgery is performed when medical treatments do not control the IOP.

Examples of agents used for treating glaucoma include: beta-blockers (e.g., timolol, betaxolol, levobetaxolol, carteolol, levobunolol, propranolol); carbonic anhydrase inhibitors (e.g., dorzolamide); alpha 1 antagonists; alpha 2 agonists (e.g., iopidine and brimonidine), miotics (e.g., pilocarpine and phosphodiesterade inhibitors; sympathomimetics (e.g., epinephrine), prostaglandin analogs (e.g., travoprost, unoprostone, and compounds set forth in U.S. Pat. Nos. 5,889,052; 5,296,504; 5,422,368; and 5,151,444)), “hypotensive lipids” (e.g., bimatoprost and compounds set forth in U.S. Pat. No. 5,352,708), and neuroprotectants (e.g., compounds from U.S. Pat. No. 4,690,931, particularly eliprodil and R-eliprodil, as set forth in U.S. Ser. No. 60/203,350 (PCT/US01/15074; PCT/US01/15169)), and appropriate compounds from WO 94/13275, including memantine. The disclosure of each of the referenced documents is incorporated herein by reference.

Ocular hypertension is a condition wherein intraocular pressure is elevated but no apparent loss of visual function has occurred. Such patients are considered to be at significant risk for the eventual development of the visual loss associated with glaucoma. If ocular hypertension is detected early and treated promptly with medications that effectively reduce elevated intraocular pressure, loss of visual function or its progressive deterioration can generally be ameliorated.

The treatment of many ophthalmic diseases and post-operative conditions require frequent administration of drugs to the ocular tissues. In almost every case, topical antibiotics are used as prophylaxis against infection after trauma or surgery. Topical corticosteroids and/or topical non-steroidal anti-inflammatory drugs (NSAID) are also routinely dispensed and are typically used at least four times per day after surgery or to treat disease. One common form of treatment is the use of drops or ointments. A topical formulation is administered by the patient or caregiver using an eye dropper or dispenser. However, a substantial disadvantage of this method of drug delivery is that the medication rapidly drains from the ocular surface into the lacrimal system through an opening in the eyelid called the punctum. Furthermore, the medication is rapidly diluted by the tears secreted by the lacrimal gland. This problem is further compounded by the patients themselves; one of the principal limitations of topical medication is poor patient compliance. The more often a patient is required to use medication, the less likely they will administer the proper dose at the proper time. Intermittent administration is also problematic because there is an initial overdosage followed by a rapid decrease in concentration due to dilution and lacrimal drainage to ineffective levels. Thus, topical treatments do not provide a continuous, prolonged delivery of medication, and the exact dosage achieved at the target tissue is unpredictable. Even further, it is often difficult for patients to administer these medications during the post-operative period due to tissue swelling, increased tearing and irritation.

Another approach for achieving localized drug delivery involves the injection of drug directly under the conjunctiva or tenon's capsule, intra-camerally or intra-vitreally. Unfortunately, this approach may require periodic injections of drug to maintain an effective drug concentration at the target site and has many potential adverse effects. A passive drug delivery system, placed under the conjunctiva or tenon's capsule by the surgeon, or in a doctor's office, at the conclusion of the case, would eliminate the need for patients to administer drugs post-operatively.

Thus, with the use of topical treatments, a continuous, prolonged delivery of medication is not achieved and the exact dosage achieved at the target tissue is unpredictable. Another problem with intermittent administration is the initial overdosage with a rapid decrease in concentration due to dilution and lacrimal drainage to ineffective levels.

Thus, there is a need to develop sustained, controlled delivery systems for many ophthalmic drugs including, but not limited to antibiotics, anti-fungals, anti-virals, anti-inflammatories, anti-glaucoma, anti-VEGF, and the like.

It is important for the treatment of ocular conditions requiring repeated application to develop a new drug delivery construct which prolongs the drugs effect and attains precision in drug dose.

In order to prolong the existence of the drug at the target site, the drug may be formulated into a slow release formulation (see, for example, Langer (1998) NATURE 392, Supp. 5-10). For example, the drug can be conjugated with polymers which, when administered to an individual, are then degraded, for example, by proteolytic enzymes or by hydrolysis, to gradually release drug into the target site. Similarly, drugs can be placed throughout insoluble matrices. Following administration, the drug then is released via diffusion out of, or via erosion of, the matrices. Alternatively, the drug can be encapsulated within a semi-permeable membrane or liposome. Following administration, the drug is released either by diffusion through the membrane or via breakdown of the membrane.

Several systems shown in U.S. patents describe large ocular inserts to continuously deliver active agents to the eye. Certain inserts disperse the drug and require removal of the carrier of the drug once the drug has been delivered. However, U.S. Pat. Nos. 3,845,201; 4,164,559 and 4,179,497 show various inserts in the form of large pellets which dispense drug over a period of time and eventually are completely eroded, and thus do not require removal after drug delivery.

U.S. Pat. No. 4,164,559 describes an ophthalmic drug delivery system comprising (a) an enzyme-extracted, chemically-modified collagen thin membrane carrier selected from the group consisting of esterified collagen and acylated collagen and having a pH in the range of 5.5-9.0 whereby the carrier is soluble in the tear fluid under physiologic conditions, and (b) an ophthalmically active drug incorporated into said carrier. U.S. Pat. No. 4,882,150 describes an ophthalmic drug delivery system, which includes at least one particle of bioerodible material, and a liquid or ointment carrier which includes ophthalmic drug to be delivered to the ocular area. The bioerodible material includes collagen. U.S. Pat. No. 5,512,301 describes collagen-containing sponges comprising an absorbable gelatin sponge, collagen, and an active ingredient are disclosed as are methods of enhancing wound healing of external and internal wounds using such sponges. U.S. Pat. No. 6,448,387 describes collagen films which rapidly dissolve at 35° C. and methods for the preparation of the collagen films and their use as a vehicle for delivering a dose of therapeutic compound to a specific tissue site. U.S. Pat. No. 5,418,222 describes a multi-layered collagen film for use in controlled release of an active ingredient, said film comprising one or two rate controlling layers and one or more drug reservoir layers, said layers comprising non-fibrillar collagen and contacting each other in a stacked conformation such that a rate controlling layer is situated at one or both ends of the stack and contacts only one other layer, said other layer being a drug reservoir layer.

These inserts have certain advantages over the liquid treatments as a more predictable dosage is obtained as there is a continuous dispensing of the drug over a period of time without rapid washout. Thus, the unit ocular inserts provide predictable dosage over a period of time without the requirement of repeated applications as required with liquid treatments.

The subconjunctival fluids will cause the collagen in the device to hydrolyze thereby continuously releasing drugs in the sub-Tenon's or subconjunctival space. As the drugs are freed, they diffuse through the conjunctiva and into the tear film constantly bathing the cornea and penetrating into the eye. Furthermore, since the device is placed on the episclera, some of the drugs will diffuse directly into the eye through the sclera; this is an advantage not found with topical application. After a pre-determined time, the collagen delivery device will completely disappear and dissolve into inactive moieties that are easily cleared by the body.

In certain configurations, using local or topical anesthesia methods, a drug delivery device containing pressure lowering medication would be surgically implanted under the conjunctiva or tenon's capsule in between the rectus muscles. It would provide long term, zero order drug delivery requiring no patient involvement. Medication would be released and diffuse through the conjunctiva into the tear film constantly bathing the cornea and penetrating into the eye. In addition, the episcleral placement of the drug delivery device is directly over the ciliary body, trabecular meshwork and Schlem's canal: these are the target site(s) for anti-glaucoma therapy. Since the sclera is 70% water, this episcleral location will also allow transscleral diffusion to these tissues, an advantage not found with topical medication.

Further ocular drug delivery implants have been described in an effort to improve and prolong drug delivery. For example, U.S. Pat. No. 3,949,750 discloses a punctual plug made of a tissue-tolerable, readily sterilizable material, such as Teflon, HEMA, hydrophilic polymer, methyl methacrylate, silicone, stainless steel or other inert metal material. It is stated that the punctual plug may be impregnated with ophthalmic medication or that the punctual plug may contain a reservoir of the ophthalmic drug. However, these plugs are often extruded and this method may inadvertently deliver drug into the lacrimal drainage system and decrease its effect.

U.S. Pat. No. 5,053,030 discloses an intracanalicular implant that can be used as a carrier or medium for distributing medications throughout the body. U.S. Pat. No. 5,469,867 discloses a method of blocking a channel, such as the lacrimal canaliculus by injecting a heated flowable polymer into the channel and allowing it to cool and solidify. The polymer may be combined with a biologically active substance that could leach out of the solid punctum once it has formed in the channel.

WO 99/37260 discloses a punctual plug made of a moisture absorbing material, which is not soluble in water, such as a modified HEMA. It is also disclosed that an inflammation inhibitor, such as heparin, may be added to the material from which the punctual plug is made.

U.S. Pat. No. 6,196,993 discloses a punctual plug containing glaucoma medication. The medication is contained in a reservoir within the plug. The reservoir is in fluid communication with a pore through which the medication is released onto the eye.

U.S. Pub. No. 2003/0143280 discloses the use of biodegradable polymer capsules for treating ophthalmic disorder including dry eye and glaucoma. The capsules are made of any biodegradable, biocompatible polymer and may contain a treating agent.

U.S. Pub. No. 2004/0013704 discloses solid or semi-solid implant compositions lacking polymeric ingredients. These implant compositions are made of lipophilic compounds and may be implanted anywhere in the eye including the punctum or lacrimal canaliculus. It is stated that the implants may contain any ophthalmic drug, including anti-glaucoma drugs.

WO 2004/066980 discloses a device for delivering a carbonic anhydrase inhibitor (CAI) to the eye over an extended period of time. In one embodiment, the device has an inner CAI-containing core and an outer polymeric layer. The outer layer may be permeable, semi-permeable, or impermeable to the drug. Where the outer layer is impermeable to the drug, it may have one or more openings to permit diffusion of the CAI.

U.S. Pub. No. 2005/0232972 discloses ocular implants to which active agents have been applied to at least one surface. In one embodiment, the implant may contain a hollow core filled with medication. In another embodiment, the medication may be applied to one or more bands of polymer material. Alternatively, a porous or absorbent material can be used to make up the entire plug or implant which can be saturated with the active agent.

WO 2006/031658 discloses lacrimal canalicular inserts including a polymer component and a therapeutic component. The polymer component may include one or more non-biodegradable polymers, one or more biodegradable polymers, or combinations thereof. The insert may comprise a matrix of a polymer component and a therapeutic component. The inserts may be coated with a substantially impermeable coating.

U.S. Pub. No. 2006/0020248 discloses an ophthalmological device for lacrimal insertion that includes a reservoir for a medication, such as a glaucoma, antimicrobial, anti-inflammatory, or dry-eye syndrome medication.

A reservoir drug-delivery device is a device that contains a receptacle or chamber for storing the drug. There are drawbacks to reservoir drug delivery devices in that they are difficult to manufacture, difficult to achieve drug content uniformity (i.e., device to device reproducibility, particularly with small ocular devices), and they carry the risk of a “dose dump” if they are punctured. In matrix drug delivery devices, the drug is dispersed throughout a polymeric matrix and is released as it dissolves or diffuses out of the matrix. Matrix devices have an advantage over reservoir devices in that they are not subject to a dose dump if punctured. A disadvantage of matrix devices is that it can be difficult to achieve zero-order drug release kinetics. Zero-order drug release or near zero-order drug release is desirable because the rate of drug release is independent of the initial concentration of the drug, thus the drug can be released at therapeutic levels over a sustained period of time. The manufacture of matrix devices can also present difficulties when the drug and the polymer are processed and extruded at elevated temperature and/or pressure as this may reduce the activity of the drug.

There are several methods to crosslink hyaluronic acid and other polysacccharides. In particular U.S. Pat. No. 6,150,461 (Takei, et. al.) describes the preparation of a copolymer in which hyaluronic acid is grafted on a polymer main chain. The main chain is a poly-L-lysine (PLL). The hyaluronic acid graft has a molecular weight of less than 100,000, preferably between 1,000 and 50,000. The purpose of the hyaluronic acid-PLL is to deliver DNA or drugs to appropriate tissues or cells containing hyaluronic acid binding sites. Hyaluronic acid is polymerized to PLL by reductive amination in a high salt buffer. In addition, there have been literature publications and patents describing chemical crosslinking of collagen and hyaluronic acid including Rehakova et. al. (1996) using starch dialdehyde and Lin et. al. (2007) using 1-ethyl-3-(3-dimethylaminopropyl-carbodiimide (EDC) and U.S. Pat. No. 8,607,044 (Schroeder, et. al., 2014) using divinyl sulfone or 1,4-butanediol diglycidyl ether (BDDE). Previous attempts to prepare such crosslinked polymers failed to achieve zero-order drug release kinetics.

Nevertheless, there exists a need for continued improvement and development of new and unique treatment constructs (e.g., films, wafers, or membranes) and methods of preparing such treatment constructs. The constructs (e.g., films, wafers, or membranes) and methods of the present invention address the above-noted deficiencies in the art by providing matrix constructs that achieve zero-order or near zero-order drug-release kinetics typically associated with reservoir constructs, but without the risk of dose dumping and the manufacturing difficulties of reservoir constructs. The present invention further provides process for preparing improved collagen-based film constructs which include therapeutic compounds for ophthalmic application. In certain embodiments, the collagen of the collagen-based film constructs is a derivatized collagen. In various embodiments, the constructs are designed to induce unidirectional flow to prevent potential loss of the drug (e.g., from flowing into the orbit and away from the intended target(s) inside the globe of the eye).

### SUMMARY OF INVENTION

The present application describes derivatized collagen-hyaluronic acid based constructs and films comprising a single drug reservoir with no rate controlling barrier or collagen-based film constructs with at least one rate controlling barrier layer, said barrier layers co-bonded to form a film construct such that the rate controlling barrier is located on one side of the construct to provide a barrier to drug diffusion in one direction and permit unidirectional delivery of said drug. In addition, the base collagen composition can be chemically derivatized to alter the overall ionic characteristics to better bind the drug component thereby altering the rate diffusion into tissues. Such constructs may be implanted, generally submucosally, to provide sustained and controlled release of drugs (i.e. active agents) for ocular applications.

One embodiment of the present invention is directed to a collagen-hyaluronic acid based film or membrane delivery system. The system comprises one or more layers comprising crosslinked, chemically derivatized collagen-hyaluronic acid; wherein the crosslinked, chemically derivatized collagen-hyaluronic acid comprises chemically derivatized collagen crosslinked with a hyaluronic acid.

Another embodiment of the present invention is directed to a method of preparing a collagen-hyaluronic acid based film or membrane delivery system. The method comprises preparing a derivatized collagen and dissolving the derivatized collagen in a buffer; mixing the derivatized collagen in a buffer with a hyaluronic acid to form a mixture; adding an active agent to the mixture to form a solution; casting the solution into a layer and allowing the layer to dry or partially dry; and exposing the dry or partially dry layer to ultraviolet irradiation in a nitrogen atmosphere to form the collagen-hyaluronic acid based film or membrane delivery system.

A further embodiment of the present invention is directed to a method for preparing a collagen-hyaluronic acid based gel. The method comprises crosslinking Poly-L-lysine hydrobromide (PLL) and hyaluronic acid to form a HA-PLL gel, by reacting hyaluronic acid with PLL dissolved in a sodium borate and borohydride buffer solution; derivatizing soluble collagen to form a derivatized collagen gel, by reacting a collagen solution at alkaline pH with an acylation agent; mixing the HA-PLL gel with the derivatized collagen gel and adjusting the pH to 9.5; adding a bifunctional acylation agent to the mixture to produce a gel; and adding 1N NaOH to the gel to adjust the pH to from about 6.8 to about 7.4.

Other embodiments will be in part described herein and in part apparent.

### BRIEF DESCRIPTION OF FIGURES

FIG. 1A and FIG. 1B illustrate examples of a rectangular curing well (also referred to herein as molds).

FIG. 2A illustrates an example of a circular curing well (also referred to herein as molds) and FIG. 2B illustrates an example of a circular film of the present invention.

FIG. 3 is an example of a multilayer collagen film system for unidirectional drug delivery.

FIG. 4 is a photograph of a rabbit model eye 30 days following implantation of a wafer of the present invention.

FIG. 5 presents the results of a release profile test of a Latanoprost-containing collagen film.

FIG. 6 presents the results of a transscleral diffusion test of an adexamethasone-fluorescein (DFL)-containing low concentration collagen film.

FIG. 7 presents the results of a transscleral diffusion test of a methotrexate gel-containing low concentration collagen film.

FIG. 8 is presents the results of a diffusion test of a gentamycin-containing collagen film.

### DETAILED DESCRIPTION OF THE INVENTION

The present invention is directed in certain parts to derivatized collagen-hyaluronic acid based constructs for sustained delivery of an active agent (i.e. drug) as well as methods for preparing the derivatized collagen-hyaluronic acid based constructs.

In certain embodiments, the film or membrane constructs are composed of collagen that is chemically derivatized to alter the ionic properties to more efficiently bind specific agents or drugs to improve sustained delivery. Drugs (i.e. active agents) are added to the films and membranes in quantities to provide uniform sustained delivery for various therapeutic time intervals. Unidirectional films or membranes are formed by applying a physical or chemical barrier to prevent diffusion of drugs and agents and only allow the drug or agent to disperse in one direction (e.g., from only one side of the film). Physical barriers may be in the form of an occlusive film bound to the base collagen construct. Chemical barriers are produced by chemically derivatizing one surface of the collagen constructs to create an occlusive barrier. The chemical barrier may be a hydrophobic surface to retard or prevent diffusion of hydrophilic drugs or agents. Diffusion flux of drugs (i.e. active agents) is dependent on ionic interactions between the collagen composition and the therapeutic agent, concentration of collagen composition and therapeutic agent, and diffusion barrier effectiveness. The ionic properties of the collagen base may be altered by derivatizing collagen with agents that alter net charge to make the collagen more anionic or more cationic. The resorption rate of the collagen films and membranes is controlled by the degree of crosslinking accomplished by controlling the time of UV irradiation.

The present invention is directed to a method of crosslinking hyaluronic acid to collagen, resulting in a soluble HA-collagen copolymer that is mixed with selected ocular drugs and then placed into wells of specific dimensions. The wells are exposed to ultraviolet irradiation (e.g., UVC) in a nitrogen atmosphere for selected time periods to form drug delivery films with desired drug delivery kinetics. Films can be produced to deliver selected drugs for time periods ranging from less than 1 minute to 6 months or more. Drugs can be trapped in the HA-collagen copolymer matrix for sustained internal or external delivery to eyes and are not ionically or covalently bound to the HA or collagen. Sustained drug delivery is especially relevant for chronic conditions such as glaucoma, uveitis or macular degeneration which require long term treatment. This invention can also be used in the immediate post-operative period to eliminate the need for patients to self-administer eyedrops (e.g. antibiotics, steroids, NSAID). Placement of the film in the anterior or posterior chamber or vitreous cavity by the surgeon at the end of the operative procedure eliminates the need for patient compliance after surgery.

HA-collagen films can be placed externally under the conjunctiva or Tenon's capsule proximately to treat diseases of the anterior segment (e.g., conjunctiva, cornea, iris, ciliary body) or distally to treat diseases of the posterior segment (e.g., vitreous, retina, choroid, or sclera). The films can also be placed intraocularly either in the anterior chamber, posterior chamber, or vitreous. Films can also be placed in the lacrimal canaliculus after dilating the punctum. Another advantage of this method is reversibility in the unlikely event of an adverse reaction, the film can be removed. The HA-collagen films can be manually placed using forceps or injected using a syringe type delivery system. Another advantage is that the construct is easily and completely reversible.

The inventors have discovered that PLL (Poly-L-lysine hydrobromide) can be used to crosslink hyaluronic acid molecules with pendent amine groups. The resultant PLL substituted hyaluronic acid can be used to crosslink similar molecules using difunctional acylation chemicals. Importantly, the PLL-hyaluronic acid molecules can be polymerized with collagen (or derivatized collagen) via covalent crosslinking between free amines on hyaluronic acid and collagen. Acylation reactions will proceed as long as the pendant free amines are in a deprotonated form (accomplished by adjusting the solution pH to the pKa of the pendant ε-amine groups, for example, about 8.5). The resultant polymerized hyaluronic acid-collagen will remain soluble until formed into drug delivery films or injectable drug delivery units.

The present invention is directed to methods of preparing degradable collagen-based film constructs which include therapeutic compounds for ophthalmic application. The method for preparing a single layer film or construct involves (i) preparing a purified solution of monoreactive-amine modified collagen (e.g., a glutaric anhydride derivatized collagen), (ii) recovering derivatized collagen precipitates and dissolving derivatized collagen precipitates in a physiological buffer (e.g., at concentrations ranging from 10 mg/mL to 100 mg/mL), (iii) adding crosslinked or non-crosslinked hyaluronic acid and adding an active drug in therapeutic dose concentrations to provide sustained release (e.g., for time periods ranging from 1 day to 6 months or more), (iv) centrifuging the drug containing derivatized collagen and hyaluronic acid to remove air bubbles, (v) casting the solution into a thin layer, wherein the solution dries and forms the film or is dehydrated to form a partially dry film, and (vi) exposing the dehydrated or partially dehydrated film to ultraviolet irradiation in a nitrogen atmosphere.

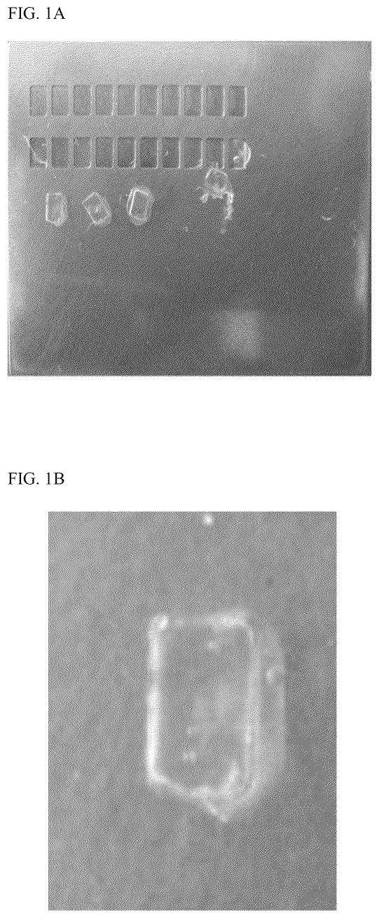

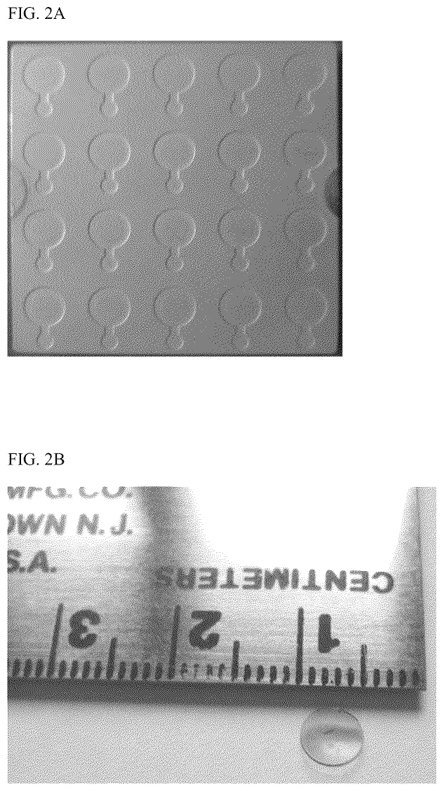

Further embodiments of the present invention are directed to methods for crosslinking hyaluronic acid to collagen (or derivatized collagen) resulting in a soluble HA-collagen copolymer that is mixed with selected ocular drugs and then placed into wells of specific dimensions and exposed to ultraviolet (e.g., UVC) irradiation in a nitrogen atmosphere for selected time periods to form drug delivery films with desired drug delivery kinetics. The wells (also referred to herein as molds) allow the collagen/HA films to be created in any size, shape or thickness. Examples of rectangular (FIG. 1A and FIG. 1B) and circular wells (FIG. 2A) are demonstrated in the Figures. The rectangular well of FIG. 1A produces films or wafers of approximate dimensions: 2 mm wide, 4 mm long, and 0.5 mm in depth. The circular well of FIG. 2A produces wafers of approximately 5 mm or 10 mm in width. For example, as shown in FIG. 2B.

In certain embodiments, the first step in the process is to polymerize hyaluronic acid with Poly-L-Lysine (PLL) to form a hyaluronic acid copolymer. The resultant copolymer can then be reacted with appropriate bifunctional acylating agents to crosslink free amine groups on the pendant PLL. Acylation reactions will proceed as long as the pendant free amines are in deprotonated form. This can be accomplished by adjusting the solution pH to the pKa of the pendant ε-amine groups (e.g., about 8.5). It is believed that the resultant polymerized hyaluronic acid will remain in soluble form (as opposed to the particulate form of Restylane and CTA) and will exhibit enhanced stability when exposed to hyaluronidase. In certain embodiments, acylation with an acylation agent alters the ionic charge and/or charge density and results in a soluble composition having a neutral pH.

The polymerized hyaluronic acid is then utilized in a similar method as described above. Derivatized collagen precipitates are recovered and dissolved in a physiological buffer, the polymerized hyaluronic acid is added to the derivatized collagen and then the active drug is added. The drug containing derivatized collagen and polymerized hyaluronic acid is centrifuged to remove air bubbles. The resulting solution is cast into a thin layer and dries and forms the film or is dehydrated to form a partially dry film. Finally, the film is exposed to ultraviolet irradiation in a nitrogen atmosphere.

In certain embodiments, the polymerized hyaluronic acid is mixed with the derivatized collagen and the pH of the mixture is adjusted to about 9.5. In certain embodiments the polymerized hyaluronic acid is mixed with the derivatized collagen and the pH of the mixture is adjusted to from about 8.5 to about 9.5, from about 8.6 to about 9.5, from about 8.7 to about 9.5, from about 8.8 to about 9.5, from about 8.9 to about 9.5, from about 9.0 to about 9.5, or from about 9.25 to about 9.5. The mixture is then combined with an acylation agent to produce a gel. Finally, a base (e.g., NaOH) is added to the gel to achieve a final pH of from about 6.8 to about 7.4. For example, a final pH of from about 6.9 to about 7.3, from about 6.9 to about 7.2, or from about 6.9 to about 7.1. The gel may be utilized as the sustained delivery construct or may be prepared as a film utilizing drying and exposure to ultraviolet light in a nitrogen atmosphere as described above.

While discussion throughout this text may be directed to films and/or gels for purposes of explaining the invention, it will be well understood that films, gels, wafers, membranes, or other suitable constructs for the application of the described collagen-hyaluronic acid based delivery system may be utilized and are included as an aspect of the present invention. Similarly, it will be understood that the constructs may be made in any suitable dimensions or shapes, as required by the particular application.

To prepare unidirectional collagen-based film constructs, the drug reservoir layer (e.g., film as described herein comprising an active agent) is prepared generally as described above. However, following casting the solution into a thin layer and partial dehydration, a second barrier layer (i.e. rate controlling barrier layer) may be applied to one side of the drug reservoir layer. The collagen composition for this layer is prepared by derivatizing collagen with an acylating agent that imparts a barrier to diffusion of the active agent. If the active agent is hydrophilic, the barrier layer should be hydrophobic. For example, soluble collagen can be derivatized with agents such as β-styrene sulfonyl chloride or polyvinyl sulfonic acid, ethylene/maleic anhydride copolymer or combinations of glutaric anhydride/β-styrene sulfonyl chloride, glutaric anhydride/polyvinyl sulfonic acid, glutaric anhydride/ethylene/maleic anhydride copolymer or other combinations of anhydrides, acid chlorides, sulfonyl chlorides, sulfonic acids producing hydrophilic derivatized collagen with anhydrides, acid chlorides, sulfonyl chlorides, or sulfonic acids producing hydrophobic derivatized collagens.

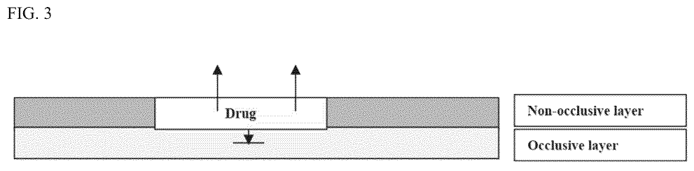

An exemplary embodiment of a multilayer collagen film system for unidirectional drug delivery is shown in FIG. 3. The top layer (“non-occlusive layer”) comprises a collagen that has been derivatized to produce a net negative charge. The bottom layer (“occlusive layer”) is a semi-occlusive layer comprising collagen derivatized to produce an ethylenically unsaturated or polymeric substituted collagen containing styrene, polyvinyl, or ethylene moieties (i.e. plastic-like properties). The net results of such a configuration is unidirectional flow out of the non-occlusive layer, away from the occlusive layer.

In one embodiment, the barrier layer is prepared by derivatizing purified collagen with glutaric anhydride and β-styrene sulfonyl chloride as previously described (U.S. Pat. No. 5,480,427). In general, the collagen solution is adjusted to pH 9.0 with 10 N and 1 N NaOH. While stirring the solution, glutaric anhydride is added at 10% (weight of collagen). For 5 minutes, the stirring is continued, and the pH maintained and 2% β-styrene sulfonyl chloride added at pH 9.0 and stirred. The pH of the solution is then adjusted to 4.3 to precipitate the derivatized collagen. The precipitate is centrifuged, washed one time, and then redissolved in phosphate buffer (0.01 M phosphate buffer, pH 7.4) to achieve a final concentration of approximately 30 mg/ml.

In some examples, the drug reservoir layer and the rate controlling barrier layer(s) may have a combined thickness of from about 0.01 to about 1 mm, from about 0.01 to about 0.9 mm, from about 0.01 to about 0.8 mm, from about 0.01 to about 0.7 mm, from about 0.01 to about 0.6 mm, from about 0.01 to about 0.5 mm, from about 0.02 to about 0.5 mm, from about 0.03 to about 0.5 mm, from about 0.04 to about 0.5 mm, or from about 0.05 to about 0.5 mm. In certain embodiments, the drug reservoir layer and the rate controlling barrier layers may have a combined thickness of from about 0.02 to about 1 mm, from about 0.02 to about 0.8 mm, from about 0.02 to about 0.6 mm, from about 0.02 to about 0.5 mm, from about 0.02 to about 0.4 mm, from about 0.02 to about 0.3 mm, or from about 0.02 to about 0.2 mm.

In some embodiments, the derivatized collagen-hyaluronic acid based film or membrane delivery system of the present invention provides sustained release of a drug or other active agent. “Sustained release” means release of a drug or other active agent from the film, membrane or other construct for a period of time that is greater than one month. In some embodiments, the release is for greater than two months. In other embodiments, the release is for greater than about 3 months, about 4 months, about 5 months, about 6 months, about 7 months, 8 about months, about 9 months, about 10 months, about 11 months, or even greater than about 12 months. In some embodiments, the release is for greater than about 6 months but for less than about one year; for greater than about 6 months but for less than about 9 months, for greater than about 6 months but for less than about 8 months, or for greater than about 6 months but for less than about 7 months.

In one embodiment, the collagen-based construct provides controlled release of a drug or other active agent. “Controlled release” means release of a drug or other active agent from the film, membrane or other construct or other construct described herein that follows zero order, or nearly zero order, release for greater than one month, greater than 2 months, greater than about 3 months, about 4 months, about 5 months, about 6 months, about 7 months, about 8 months, about 9 months, about 10 months, about 11 months, or even greater than about 12 months. In some embodiments, the release is for greater than about 6 months but for less than about one year; for greater than about 6 months but for less than about 9 months; for greater than about 6 months but for less than about 8 months; or for greater than about 6 months but for less than about 7 months. In some embodiments, controlled release includes an initial bolus release on day 1 or on about days 1 to 3. In other embodiments, the controlled release does not include a bolus release on day 1 so that zero order, or nearly zero order, release begins on day 1. In still other embodiments, the controlled release does not include a bolus release after day 3, so that zero order, or nearly zero order, release begins on about day 4. A controlled release is a sustained release, but a sustained release is not necessarily a controlled release.

In some embodiments, the collagen-hyaluronic acid based film or membrane delivery system of the present invention provides sustained release and/or controlled release of a drug or other active agent that is sparingly soluble in aqueous solution but soluble in alcohols and other water miscible solvents mixed into the composition.

Hyaluronic acid can be prepared by fermentation. Molecular weights may range from as low as about 25,000 Daltons to more than about 3 million Daltons. For example, the molecular weight may be from about 25,000 to about 2.5 million Daltons, from about 50,000 to about 2 million Daltons, from about 50,000 to about 1.75 million Daltons, from about 50,000 to about 1.5 million Daltons, from about 50,000 to about 1.25 million Daltons, or from about 50,000 to about 1 million Daltons. In certain embodiments, the molecular weight may be from about 150,000 to about 2 million Daltons.

PLL hydrobromide may have a molecular weight of from about 500 Daltons (e.g., Sigma Catalog Number P 8954) to more than about 300,000 Daltons (e.g., Sigma Catalog Number P 5899). In certain embodiments, the PLL hydrobromide may have a molecular weight of from about 500 to about 250,000 Daltons, from about 500 to about 200,000 Daltons, from about 500 to about 150,000 Daltons, or from about 500 to about 100,000 Daltons.

Collagen can be derived from bovine, porcine, fish, human, or recombinant human sources. Acylation reactions to derivatize soluble and insoluble collagen and have been described by DeVore, et al. in a series of patents (U.S. Pat. Nos. 4,713,446, 4,851,513, 4,969,912, 5,067,961, 5,104,957, 5,201,764, 5,219,895, 5,332,809, 5,354,336, 5,476,515, 5,480,427, 5,631,243, and 6,161,544) which are incorporated herein by reference.

In some embodiments, the concentration of the derivatized collagen in the solution comprising the derivatized collagen, hyaluronic acid, and active agent is from about 15 mg/mL to about 100 mg/mL. In other embodiments the concentration is from about 25 mg/mL to about 60 mg/mL. In still other embodiments, the concentration is from about 30 mg/mL to about 45 mg/mL.

In some embodiments, the concentration of acylating agent used to prepare derivatized collagen, as a percentage of the total weight of the base collagen, may be selected such that it minimally increases the net negative charge. For example, in one embodiment, the concentration of acylating agent may be less than about 5%, less than about 4%, less than about 3%, less than about 2%, or less than about 1% of the weight of the collagen that is derivatized. In other embodiments, the concentration of acylating agent, as a percentage of the total weight of the base collagen, may be selected such that it moderately increases the net negative charge. For example, the concentration of acylating agent may be less than about 20%, less than about 15%, less than about 10%, less than about 9%, less than about 8%, less than about 7%, or less than about 6% of the weight of the collagen that is derivatized. In still further embodiments, the concentration of acylating agent, as a percentage of the total weight of the base collagen, may be selected such that it maximally increases the net negative charge. For example, the concentration of acylating agent may be about 25% or greater, about 30% or greater, about 35% or greater, about 40% or greater, about 45% or greater, or about 50% or greater of the weight of the collagen that is derivatized.

In certain embodiments, acylating agents may be used to alter the net charge and charge density of intact tissue proteins. An increase in net negative charge density will increase ionic binding of agents with a net positive charge. Certain agents can be used to change the net charge from positive to negative. These agents include, but are not limited to, anhydrides including maleic anhydride, succinic anhydride, glutaric anhydride, citractonic anhydride, methyl succinic anhydride, itaconic anhydride, methyl glutaric anhydride, dimethyl glutaric anhydride, phthalic anhydride, and many other such anhydrides. Acid chlorides include, but are not limited to, oxalyl chloride, malonyl chloride, and many others. Sulfonyl chlorides include, but are not limited to, chlorosulfonylacetyl chloride, chlorosulfonylbenzoic acid, 4-chloro-3-(chlorosulfonyl)-5-nitroebnzoic acid, 3-(chlorosulfonyl)-P-anisic acid, and others. Sulfonic acids include, but are not limited to, 3-sulfoebnzoic acid and others. In some embodiments, the acylating agent may comprise a combination of one or more of the above-described acylating agents.

Certain agents can change the net charge from one positive to two negatives per reacted site. Specific agents include, but are not limited to, 3,5-dicarboxybenzenesulfonyl chloride and others.

Certain agents can be used to change the net charge from positive to neutral per reacted site. Specific agents include, but are not limited to, anhydrides including acetic anhydride, chloroacetic anhydride, propionic anhydride, butyric anhydride, isobutyric anhydride, isovaleric anhydride, hexanoic anhydride, and other anhydrides; acid chlorides including acetyl chloride, propionyl chloride, dichloropropionyl chloride, butyryl chloride, isobutyryl chloride, valeryl chloride, and others; sulfonyl chlorides including, but not limited to, ethane sulfonyl chloride, methane sulfonyl chloride, 1-butane sulfonyl chloride, and others.

Certain agents can be used to change the net charge from one positive to two positives per reacted site. Specific agents include, but are not limited to, 4,6-diamino-2-methylthiopyrimidine-5-sulfonic acid, and others.

Bifunctional or multifunctional acylating agents may be selected from the group consisting of the following coupling agents which have two or three groups which react with amines but do not react with carboxyl groups. Such coupling agents include di- and tri-carboxylic acid halides, di- and tri-sulfonyl halides, di- and tri-anhydrides, di- and tri-reactive active esters and coupling agents containing at least two groups of the carboxylic acid halide, sulfonyl halide, anhydride or active ester type. Aromatic and aliphatic di- and tri-carboxylic acid halides may be selected from the group consisting of d-camphoric diacid chloride; 4-p-(o-chlorocarbonylbenzoyl)phenyl]butyryl chloride; furan-3,5-dicarboxylic chloride; fumaryl chloride; glutaryl chloride; succinyl chloride; sebacoyl chloride; isophthaloyl chloride; terephthaloyl chloride; 4-bromoisophthaloyl chloride; diglycolic diacid chloride; 1,1-cyclohexanediacetyl chloride; 2,2-dimethylglutaryl chloride; thioglycolic acid dichloride; nitrilotriacetyl chloride; beta-methylcarballylic acid trichloride; hexadecanedioic acid dichloride; malonic acid dichloride; acetone dicarboxylic acid dichloride; oxydiacetyl chloride benzene-1,3,5-tricarbonyl chloride; 4-chlorocarbonylphenoxyacetyl chloride; homophthaloyl chloride; 4,4′-diphenyletherdicarboxylic acid dichloride; 4,4′-diphenylthioetherdicarboxylic acid dichloride; 4,4′-diphenylsulfonedicarboxylic acid dichloride; acetylene dicarboxylic acid dichloride; cyclohexane-1,4-dicarboxylic acid dichloride; trans-3,6-endomethylene-1,2,3,6-tetrahydrophthaloyl chloride; 4,4′-dithiodibutyryl chloride; diphenylmethane-4,4′-bis(oxyacetyl) chloride; N-(4-chlorocarbonylphenyl)anthranyloyl chloride; 1,3-benzenebisoxyacetyl chloride; pyridine-3,5-dicarboxylic acid dichloride; pyridine-2,5-dicarboxylic acid dichloride; pyridine-2,4-dicarboxylic acid dichloride; pyrazine-2,3-dicarboxylic acid dichloride; and pyridine-2,6-dicarboxylic acid dichloride; ethyleneglycol bis-4-chlorocarbonylphenyl)ether; diethyleneglycol bis-4-chlorocarbonylphenyl)ether; bis-4-chlorocarbonyl-2-tolyl)thioether; and N-chlorocarbonylmethyl-N-methylglutaramic acid chloride.

Aromatic and aliphatic di- or trisulfonyl halides may be selected from the group consisting of para-fluorosulfonylbenzenesulfonyl chloride; 1,3,5-benzenetrisulfonyl chloride; 2,6-naphthalenedisulfonyl chloride; 4,4′-biphenyl disulfonyl chloride; 1,10-decane-disulfonyl chloride; and 4,4′-trans-stilbenedisulfonyl chloride.

Di- and trianhydride coupling agents may be selected from the group consisting of 1,2,4,5-benzenetetracarboxylic dianhydride; 3,4,9,10-perylene tetracarboxylic dianhydride; 3,3′,4,4′-benzophenonetetracarboxylic dianhydride; 1,2,7,8-naphthalenetetracarboxylic dianhydride; pyromellitic dianhydride; 2,3,4,5-tetrahydrofurantetracarboxylic acid dianhydride; mellitic trianhydride; 1,2,3,4-cyclobutanetetracarboxylic dianhydride; bicyclo[2,2,2]oct-7-ene-2,3,5,6-tetracarboxylic dianhydride; cyclopentanetetracarboxylic dianhydride; ethylenediaminetetraacetic dianhydride; and diethylenetriaminepentaacetic dianhydride.

Coupling agents containing combinations of amine-reactive groups may be selected from the group consisting of 5-chlorosulfonyl-ortho-anisic acid chloride; 2-chloro-5-fluorosulfonylbenzoyl chloride; 4-chlorosulfonylphenoxyacetyl chloride; meta-fluorosulfonylbenzoyl chloride; and trimellitic anhydride acid chloride.

The concentration of the coupling agent is dependent upon many factors including the reactivity of the coupling agent. In general, however, the amount of the coupling agent is from about 1 to about 30% (w/v or v/v) of coupling agent per unit volume of derivatized collagen. For example, from about 1% to about 29%, from about 1% to about 28%, from about 1% to about 27%, from about 1% to about 26%, from about 1% to about 25%, from about 2% to about 25%, from about 3% to about 25%, from about 4% to about 25%, from about 5% to about 25%, from about 6% to about 25%, from about 7% to about 25%, from about 8% to about 25%, from about 9% to about 25%, from about 10% to about 25%, from about 10% to about 24%, from about 10% to about 23%, from about 10% to about 22%, from about 10% to about 21%, or from about 10% to about 20%.

In order to limit the degree of coupling, in certain embodiments, the mixture of collagen (e.g., derivatized collagen) and hyaluronic acid may contain purified collagen in a concentration of from about 0.05 to about 0.3 wt %, from about 0.10 to about 0.3 wt %, from about 0.11 to about 0.3 wt %, from about 0.12 to about 0.3 wt %, from about 0.13 to about 0.3 wt %, from about 0.14 to about 0.3 wt %, or from about 0.15 to about 0.3 wt %. In further embodiments, the solution comprising the mixture of collagen (e.g., derivatized collagen) and hyaluronic acid and added active agent may contain purified collagen in a concentration of from about 0.05 to about 0.3 wt %, from about 0.10 to about 0.3 wt %, from about 0.11 to about 0.3 wt %, from about 0.12 to about 0.3 wt %, from about 0.13 to about 0.3 wt %, from about 0.14 to about 0.3 wt %, or from about 0.15 to about 0.3 wt %.

In certain embodiments, the pH of the mixture (i.e. mixture of collagen and hyaluronic acid or mixture of collagen and HA-PLL) and/or solution (i.e. mixture of collagen, hyaluronic acid, and added active agent or mixture of collagen, HA-PLL, and added active agent) may be maintained throughout the coupling reaction at a desired pH level by addition of a dilute base (e.g., sodium hydroxide). For example, the pH of the mixture and/or solution may be maintained throughout the coupling reaction at a pH of from about 8 to about 11, from about 8.5 to about 11, from about 8.5 to about 10.5, from about 9 to about 40.5, or from about 9.0 to about 10.0. In one embodiment, the pH is maintained at about 9.5. In this manner, in the example of a solution comprising HA-PLL, almost all of the lysine epsilon amino groups present on the HA-PLL molecules and derivatized collagen molecules are freed from their protonated form, and become capable of reaction with either the coupling agent.

A variety of drugs (i.e. active agents) are suitable for incorporation in the collagen constructs, consistent with their known dosages and uses and are described in further detail below

Ophthalmic drugs suitable for the present invention that exhibit low water or aqueous liquid solubility include those selected from the group consisting of anti-inflammatory agents, anti-infective agents (including antibacterial, antifungal, antiviral, antiprotozoal agents), anti-allergic agents, antihistamines, antiproliferative agents, anti-angiogenic agents, anti-oxidants, antihypertensive agents, neuroprotective agents, cell receptor agonists, cell receptor antagonists, immunomodulating agents, immunosuppressive agents, intraocular (“IOP”) lowering agents, carbonic anhydrase inhibitors, cholinesterase inhibitor miotics, prostaglandins and prostaglandin receptor agonists, prostaglandin F derivatives, prostaglandin F2a receptor antagonists, cyclooxygenase-2 inhibitors, muscarinic agents, and combinations thereof.

Non-limiting examples of the glucocorticosteroids, which may be suitable drugs for the present invention, include: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fiuprednidene acetate, fluprednisolone, flurandrenolide, fluticasone propionate, formocortal, halcinonide, halobetasol propionate, halometasone, halopredone acetate, hydrocortarnate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, their physiologically acceptable salts, derivatives thereof, combinations thereof, and mixtures thereof.

Non-limiting examples of the non-steroidal anti-inflammatory drugs (“NSAIDs”) are: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), .epsilon.-acetamidocaproic acid, S-(5′-adenosyl)-L-methionine, 3-amino-4-hydroxybutyric acid, amixetrine, bendazac, benzydamine, .alpha.-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, their physiologically acceptable salts, combinations thereof, and mixtures thereof.

Non-limiting examples of antibiotics include doxorubicin; aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin SV, rifapentine, rifaximin), .beta.-lactams (e.g., carbacephems (e.g., loracarbef)), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefinenoxime, cefodizime, cefonicid, cefoperazone, ceforamide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefinetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin G benzathine, penicillin G benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, ticarcillin), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin S, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin).

Other examples of antibiotics are the synthetic antibacterials, such as 2,4-diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, gatifloxacin, grepafloxacin, lomefloxacin, miloxacin, moxifloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benzylsulfamide, chloramine-B, chloramine-T, dichloramine T, n²-formylsulfisomidine, n⁴-.beta.-D-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, 4-sulfanilamidosalicylic acid, n⁴-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (e.g., clofoctol, hexedine, methenamine, methenamine anhydromethylene citrate, methenamine hippurate, methenamine mandelate, methenamine sulfosalicylate, nitroxoline, taurolidine, xibomol).

Non-limiting examples of immunosuppressive agents include dexamethasone, cyclosporin A, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, tegafur), fluocinolone, triaminolone, anecortave acetate, fluorometholone, medrysone, and prednisolone.

Non-limiting examples of antifungal agents include polyenes (e.g., amphotericin B, candicidin, dermostatin, filipin, fungichromin, hachimycin, hamycin, lucensomycin, mepartricin, natamycin, nystatin, pecilocin, perimycin), azaserine, griseofulvin, oligomycins, neomycin undecylenate, pyirolnitrin, siccanin, tubercidin, viridin, allylamines (e.g., butenafine, naftifine, terbinafine), imidazoles (e.g., bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole), acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, and zinc propionate.

Non-limiting examples of antiviral agents include acyclovir, carbovir, famciclovir, ganciclovir, penciclovir, and zidovudine.

Non-limiting examples of antiprotozoal agents include pentamidine isethionate, quinine, chloroquine, and mefloquine.

Exemplary drugs for glaucoma treatment include beta-blockers (e.g., timolol, betaxolol, levobetaxolol, carteolol, levobunolol, propranolol), carbonic anhydrase inhibitors (e.g., brinzolamide and dorzolamide), alpha 1 antagonists (e.g., nipradolol), alpha 2 agonists (e.g. iopidine and brimonidine), miotics (e.g., pilocarpine), prostaglandin analogs (e.g., latanoprost (13,14-dihydro-17-phenyl-18,19,20-trinor-prostaglandin F2a-1-isopropyl ester), travoprost, unoprostone, and compounds set forth in U.S. Pat. Nos. 5,889,052; 5,296,504; 5,422,368; and 5,151,444), “hypotensive lipids” (e.g., bimatoprost and compounds set forth in U.S. Pat. No. 5,352,708), and neuroprotectants (e.g., compounds from U.S. Pat. No. 4,690,931, particularly eliprodil and R-eliprodil, as set forth in a pending application U.S. Ser. No. 60/203,350 (PCT/US01/15074; PCT/US01/15169), and appropriate compounds from WO 94/13275, including memantine. The disclosure of each of the referenced documents is incorporated herein by reference.

Exemplary drugs (i.e. active agents) with low solubility in aqueous solution may also include steroids, certain antibiotics as described above, and neurotrophins. Other exemplary drugs include, but are not limited to, Auralagan (benzocaine plus antipyrine), Ciprodex (ciprofloxacin and dexamethasone), Cortisporin (hydrocortisone, polymiyxin, and neosporin), Triethanolamine, and Ofloxacin (Floxin).

Other exemplary agents include corticosteroid or other anti-inflammatory drugs as noted above (e.g., an NSAIDs), decongestants (e.g., vasoconstrictor), mucous thinning agents (e.g., an expectorant or mucolytic), agents that prevents or modifies an allergic response (e.g., an antihistamine, cytokine inhibitor, leucotriene inhibitor, IgE inhibitor, immunomodulator), anesthetic agents with or without a vasoconstriction agents (e.g. Xylocaine with or without Epinephrine), analgesic agents, hemostatic agents to stop bleeding, anti-proliferative agents, cytotoxic agents e.g. stem cells, genes or gene therapy preparations, viral vectors carrying proteins or nucleic acids such as DNA or mRNA coding for important therapeutic functions or substances, cauterizing agents e.g. silver nitrate, etc.

Drugs are added to the above-described crosslinked HA-collagen solutions or gels or to the drug reservoir films or film layers at concentrations suitable to provide sustained release for appropriate therapeutic activity for desired delivery time periods. For example, as described in the Examples, Latanoprost (Xalatan®), a medication that reduces the pressure inside the eye, can be added to reconstituted, HA or derivatized collagen solutions to produce a film construct providing a 3-month daily delivery of at least about 1.5 μg/day.

In certain embodiments, the extended therapeutic concentration of the active agent is at least about 150 μg, at least about 175 μg, at least about 200 μg, at least about 225 μg, at least about 250 μg, at least about 275 μg, at least about 300 μg, at least about 325 μg at least about 350 μg, at least about 400 μg, at least about 450 μg, or at least about 500 μg per film or membrane. In various embodiments, the extended therapeutic concentration provides effective sustained, zero-order delivery dosages for from about 1 week to about 6 months, from about 2 weeks to about 6 months, from about 2 weeks to about 5 months, from about 2 weeks to about 4 months, from about 2 weeks to about 3 months, from about 2 weeks to about 2 months, or from about 2 weeks to about 1 month.

In certain other embodiments, the extended therapeutic concentration of the active agent is selected such that a certain effective dosage per day can be achieved over a set period of time. For example, to provide an effective dosage of about 1.5 μg per day for at least about 30 days, about 1.5 μg per day for at least about 60 days, about 1.5 μg per day for at least about 90 days, or about 1.5 μg per day for at least about 180 days, or

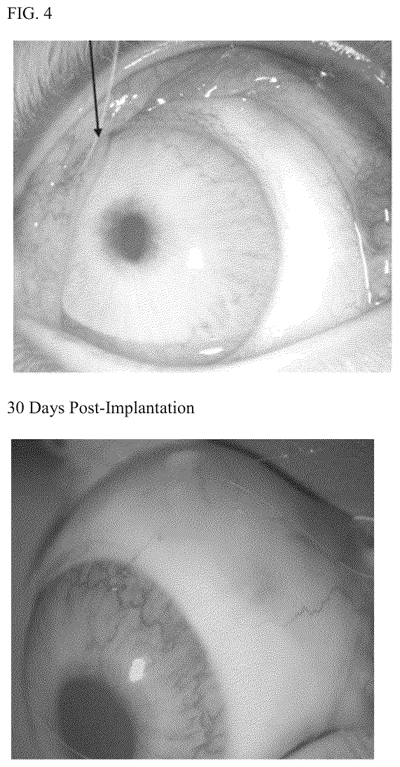

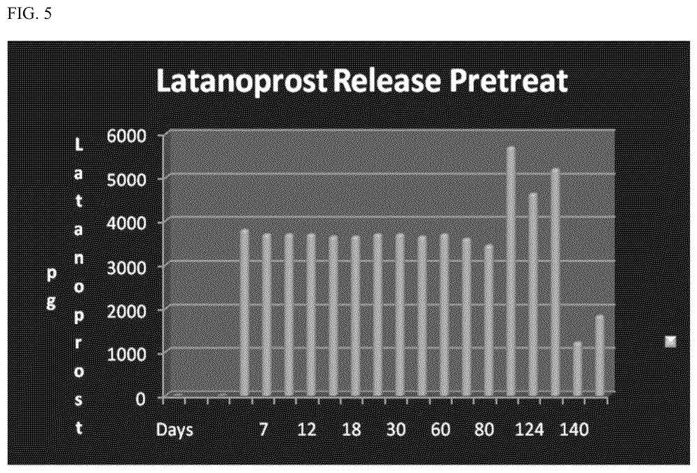

In still other embodiments, the extended therapeutic concentration results in film that releases the drug with an initial bolus until about day 1, day 2, or day 3, followed by controlled release for at least about 3, 4, 5, 6, 7, 8, 9, 10, 11, or 12 months. In still further embodiments, the controlled release is for at least 6 months, but not for more than about 7, 8, 9, 10, 11, or 12 months. Alternating views of a photograph of a rabbit model eye 30 days following implantation of a wafer of the present invention is shown in FIG. 4, with an arrow directed at the implant. An example release profile of a Latanoprost therapeutic is shown in FIG. 5.

Other drug reservoir films or film layers can similarly be prepared to provide delivery of therapeutically effective of amounts of each drug. In some embodiments, the delivery systems are prepared by forming films or membranes of acylated collagen containing extended therapeutic concentrations of selected drugs and exposing the drug containing compositions to ultraviolet irradiation in a nitrogen atmosphere for time periods ranging from about 2 minutes to about 20 minutes. For example, from about 3 to about 20 minutes, from about 3 to about 19 minutes, from about 4 to about 19 minutes, from about 5 to about 19 minutes, from about 5 to about 18 minutes, from about 5 to about 17 minutes, from about 5 to about 16 minutes, from about 5 to about 15 minutes, from about 5 to about 14 minutes, from about 5 to about 13 minutes, from about 5 to about 12 minutes, from about 5 to about 11 minutes, or from about 5 to about 10 minutes. In some embodiments the exposure is for time periods ranging from about 14 to about 19 minutes, from about 15 to about 19 minute, from about 16 to about 18 minutes, about 15, about 16, about 17, about 18, or about 19 minutes.

Exposure to ultraviolet irradiation (e.g., UVC) in a nitrogen atmosphere is conducted to produce collagen-hyaluronic acid films or membranes with defined resorption characteristics ranging from about 1 month to about 6 months, the resorption characteristics being controlled by time of exposure to ultraviolet light in a nitrogen atmosphere. For example, exposure for about 5 minutes can be used to produce films or membranes resorbing or degrading before about 1 month, exposure for about 18 minutes can be used to produce films or membranes resorbing or degrading after about 6 months, and exposure for about 12 minutes can be used to produce films or membranes resorbing or degrading after about 3-4 months.

In some embodiments, the UV-crosslinked collagen construct is further treated by incubating it in a solution, for example a saline solution. In some embodiments, the solution treatment is for about 1 day, 2 days, 3 days, 4 days, 5 days, 6 days, 7, days, about 1 week, about 2 weeks, or about 3 weeks. In some embodiments the solution treatment results in collagen construct that releases drug with zero order kinetics or with near zero order kinetics.

Embodiment 1 is directed to a collagen-hyaluronic acid based film or membrane delivery system comprising:
one or more layers comprising crosslinked, chemically derivatized collagen-hyaluronic acid;
wherein the crosslinked, chemically derivatized collagen-hyaluronic acid comprises chemically derivatized collagen crosslinked with a hyaluronic acid.

Embodiment 2 is directed to the film or membrane delivery system of Embodiment 1, comprising:
two or more layers of crosslinked, chemically derivatized collagen-hyaluronic acid;
wherein at least one layer is a rate controlling layer having multiple surfaces composed of derivatized collagen on at least one surface of the multiple surfaces.

Embodiment 3 is directed to the film or membrane delivery system of Embodiment 1, wherein the collagen is derivatized with an acylating agent to alter the ionic charge and charge density.

Embodiment 4 is directed to the film or membrane delivery system of Embodiment 3, wherein the acylating agent is selected from the group consisting of anhydrides including maleic anhydride, succinic anhydride, glutaric anhydride, citractonic anhydride, methyl succinic anhydride, itaconic anhydride, methyl glutaric anhydride, dimethyl glutaric anhydride, phthalic anhydride; acid chlorides including oxalyl chloride, malonyl chloride; sulfonyl chlorides including chlorosulfonylacetyl chloride, chlorosulfonylbenzoic acid, 4-chloro-3-(chlorosulfonyl)-5-nitrobenzoic acid, and 3-(chlorosulfonyl)-P-anisic acid; sulfonic acids including 3-sulfobenzoic acid; and combinations thereof.

Embodiment 5 is directed to the film or membrane delivery system of Embodiment 4, wherein the concentration of acylating agent, based on the weight of chemically derivatize collagen, is less than about 20%, less than about 15%, less than about 10%, less than about 9%, less than about 8%, less than about 7%, or less than about 6%, less than about 5%, less than about 4%, less than about 3%, less than about 2%, or less than about 1%.

Embodiment 6 is directed to the film or membrane delivery system of Embodiment 3, wherein the concentration of acylating agent, based on the weight of chemically derivatize collagen, is about 25% or greater, about 30% or greater, about 35% or greater, about 40% or greater, about 45% or greater, or about 50% or greater.

Embodiment 7 is directed to the film or membrane delivery system of Embodiment 1, wherein collagen is altered to increase the net positive charge by reacting the collagen solution with an acylating agent comprising 4,6-diamino-2-methylthiopyrimidine-5-sulfonic acid.

Embodiment 8 is directed to the film or membrane delivery system of Embodiment 7, wherein the concentration of acylating agent, based on the weight of chemically derivatize collagen, is less than about 20%, less than about 15%, less than about 10%, less than about 9%, less than about 8%, less than about 7%, or less than about 6%, less than about 5%, less than about 4%, less than about 3%, less than about 2%, or less than about 1%.

Embodiment 9 is directed to the film or membrane delivery system of Embodiment 7 wherein the concentration of acylating agent, based on the weight of chemically derivatize collagen, is about 25% or greater, about 30% or greater, about 35% or greater, about 40% or greater, about 45% or greater, or about 50% or greater.

Embodiment 10 is directed to the film or membrane delivery system of Embodiment 1, wherein the film or membrane delivery system is prepared by forming films or membranes of acylated collagen containing extended therapeutic concentrations of an active agent and exposing the active agent containing compositions to ultraviolet irradiation in a nitrogen atmosphere for time periods ranging from about 2 minutes to about 14 minutes, preferably about 5 minutes to about 12 minutes.

Embodiment 11 is directed to the film or membrane delivery system of Embodiment 10, wherein the collagen-hyaluronic acid films or membranes delivery system has defined resorption characteristics from about 2 weeks to about 4 months.

Embodiment 12 is directed to the film or membrane delivery system of Embodiment 1, comprising extended therapeutic concentrations of drugs selected from the group consisting of tetracycline, chlortetracycline, bacitracin, neomycin, polymyxin, gramicidin, oxytetracycline, chloramphenicol, gentamycin, penicillin, kanamycin, amikacin, sisomicin, tobramycin, garamycin, ciprofloxacin, norfloxacin and erythromycin; antibacterials such as sulfonamides, sulfacetamide, sulfamethizole and sulfisoxazole; antivirals, including idoxuridine; and other antibacterial agents such as nitrofurazone and sodium propionate; anti-allergenics such as antazoline, methapyriline, chlorpheniramine, pyrilamine and prophenpyridamine; anti-inflammatories such as cortisone, hydrocortisone, hydrocortisone acetate, dexamethasone, dexamethasone 21-phosphate, fluocinolone, medrysone, prednisolone, methylprednisolone, prednisolone 21-phosphate, prednisolone acetate, fluorometholone, betamethasone, fluocortolone, indomethacin and triamcinolone; decongestants such as phenylephrine, naphazoline and tetrahydrozoline; miotics and anti-cholinesterase's such as pilocarpine, eserine salicylate, carbachol, di-isopropyl fluorophosphate, phospholine iodide, echothiophate, physostigmine and demecarium bromide; mydriatics such as atropine sulfate, cyclopentolate, homatropine, scopolamine, tropicamide, eucatropine, and hydroxyamphetamine; sympathomimetics such as epinephrine and immunosuppressants such as cyclosporin and azathioprine combined with acylated collagen at neutral pH; and combinations thereof.

Embodiment 13 is directed to the film or membrane delivery system of Embodiment 1 wherein the films or membranes comprise extended therapeutic concentrations of ophthalmic drugs for treatment of glaucoma selected from the group consisting of timolol, betaxolol, levobetaxolol, carteolol, levobunolol, propranolol), carbonic anhydrase inhibitors (e.g., brinzolamide and dorzolamide), al antagonists (e.g., nipradolol), α2 agonists (e.g. iopidine and brimonidine), miotics (e.g., pilocarpine), prostaglandin analogs (e.g., latanoprost, travoprost, unoprostone, others, and combinations thereof.

Embodiment 14 is directed to a method of using the film or membrane delivery system of Embodiment 13 for treatment of glaucoma, comprising placing the film or membrane intraocularly in the anterior chamber, posterior chamber, or vitreous or placing the film or membrane in the lacrimal canaliculus after dilating the punctum.

Embodiment 15 is directed to the film or membrane delivery system of Embodiment 1, wherein the films or membranes further comprise an extended therapeutic concentration of an active agent, wherein the concentration is at least about 150 μg, at least about 175 μg, at least about 200 μg, at least about 225 μg, at least about 250 μg, at least about 275 μg, at least about 300 μg, at least about 325 μg at least about 350 μg, at least about 400 μg, at least about 450 μg, or at least about 500 μg per film or membrane to provide an effective sustained, zero-order delivery dosage for from about 1 week to about 6 months, from about 2 weeks to about 6 months, from about 2 weeks to about 5 months, from about 2 weeks to about 4 months, from about 2 weeks to about 3 months, from about 2 weeks to about 2 months, or from about 2 weeks to about 1 month.

Embodiment 16 is directed to the film or membrane delivery system of Embodiment 1 wherein the films or membranes further comprise extended therapeutic concentrations of an active agent selected from the group consisting of Auralagan (benzocaine plus antipyrine), Ciprodex (ciprofloxacin and dexamethasone), Cortisporin (hydrocortisone, polymiyxin, and neosporin), Triethanolamine, and Ofloxacin (Floxin), and combinations thereof.

Embodiment 17 is directed to a method of preparing a collagen-hyaluronic acid based film or membrane delivery system, comprising:
preparing a derivatized collagen and dissolving the derivatized collagen in a buffer;
mixing the derivatized collagen in a buffer with a hyaluronic acid to form a mixture;
adding an active agent to the mixture to form a solution;
casting the solution into a layer and allowing the layer to dry or partially dry; and
exposing the dry or partially dry layer to ultraviolet irradiation in a nitrogen atmosphere to form the collagen-hyaluronic acid based film or membrane delivery system.

Embodiment 18 is directed to the method of Embodiment 17, wherein the hyaluronic acid is a crosslinked hyaluronic acid prepared by crosslinking hyaluronic acid and a polymer.

Embodiment 19 is directed to the method of Embodiment 18, wherein Poly-L-lysine hydrobromide (PLL) is crosslinked with hyaluronic acid to form the crosslinked hyaluronic acid HA-PLL.

Embodiment 20 is directed to the method of Embodiment 17, wherein mixing the derivatized collagen in a buffer with a hyaluronic acid comprises crosslinking the derivatized collagen and hyaluronic acid with an acylating agent.

Embodiment 21 is directed to a method for preparing a collagen-hyaluronic acid based gel, comprising:
crosslinking Poly-L-lysine hydrobromide (PLL) and hyaluronic acid to form a HA-PLL gel, by reacting hyaluronic acid with PLL dissolved in a sodium borate and borohydride buffer solution;
derivatizing soluble collagen to form a derivatized collagen gel, by reacting a collagen solution at alkaline pH with an acylation agent;
mixing the HA-PLL gel with the derivatized collagen gel and adjusting the pH to 9.5;
adding a bifunctional acylation agent to the mixture to produce a gel; and
adding 1N NaOH to the gel to adjust the pH to from about 6.8 to about 7.4.

Embodiment 22 is directed to the method of Embodiment 21 wherein the hyaluronic acid molecular weight is 1 million daltons and ranges from 150,000 to 2 million daltons.

Embodiment 23 is directed to the method of Embodiment 21 wherein the soluble collagen is derivatized with an acylation agent selected from the group consisting of sulfonic acids, sulfonyl chlorides, acid chlorides, and combinations thereof.

Embodiment 24 is directed to the method of Embodiment 23 wherein the soluble collagen is extracted, isolated, and purified from bovine, porcine, or human collagen

Embodiment 25 is directed to a HA-PLL-Collagen copolymer prepared according to Embodiment 21 wherein the hyaluronic acid concentration is from about 1 to about 3 wt % and the collagen concentration is from about 1 to about 5 wt %.

Embodiment 26 is directed to the method of Embodiment 21, further comprising mixing an active agent with the HA-PLL gel and derivatized collagen gel.

Embodiment 27 is directed to the method of Embodiment 21, further comprising placing the gel into a mold to form a film and exposing the gel to ultraviolet irradiation in a nitrogen atmosphere.

Embodiment 28 is directed to the method of Embodiment 27 wherein the film is deposited manually in a selected ocular location for sustained drug delivery for a desired period of time.

Embodiment 29 is directed to the method of Embodiment 28, wherein depositing manually comprises the use of forceps or a syringe type delivery system.

Embodiment 30 is directed to the method of Embodiment 29, wherein the procedure is reversible and the construct is removed using the forceps or syringe type delivery system.

Although the present invention has been described herein as related to ocular applications (e.g., treat ocular tissue defects and deficiencies), the described constructs are also applicable to other fields. For example, the constructs may be used as a topical film to treat dermal defects and infections.

The present invention provides a number of advantages. For example, the present techniques and collagen film compositions facilitate an improved approach for sustained delivery of active agents where a precise dose and accurate placement are required. The dose can be adjusted to any desired amount, i.e., by modifying the concentration of compound in the film or the size of the film, and the solid nature of the film allows its placement at any site in the body which can be reached by surgical techniques.

The features and other details of the invention will now be more particularly described and pointed out in the following examples describing preferred techniques and experimental results. These examples are provided for the purpose of illustrating the invention and should not be construed as limiting, particularly with respect to the exemplified drug.

### EXAMPLES

### Example 1. Sustained Release from Multi-Directional Collagen-Based Films

### Preparation of Drug Reservoir Collagen-Based Films

Purified, pepsin digested collagen at 3 mg/mL was obtained from Advanced Biomatrix, Inc. and derivatized with glutaric anhydride as previously described (U.S. Pat. Nos. 5,631,243 and 5,492,135, incorporated herein by reference). The collagen solution was adjusted to pH 9.0 with 10 N and 1 N NaOH. While stirring the solution, glutaric anhydride was added at 10% (based on the weight of collagen). For 5 minutes, the stirring continued, and the pH was maintained.

The pH of the solution was then adjusted to 4.3 with 6 N and 1 N HCl to precipitate the derivatized collagen. The precipitate was centrifuged at 3500 rpm for 20 minutes, washed in pyrogen-free deionized water, and then redissolved in a phosphate buffer (0.01 M phosphate buffer, pH 7.4) to achieve a final concentration of approximately 30 mg/ml. Hyaluronic acid gels were added to provide a concentration of 10-40% in the collagen-hyaluronic composition.

### Preparation of Collagen Films Containing Active Agents

To prepare collagen-hyaluronic acid films, 500 μg of an active agent was mixed with 4 mL of derivatized collagen-hyaluronic acid as prepared above. The mixture was centrifuged at 3500 rpm for 10 minutes if air bubbles were observed. The mixture was then poured into 35 mm wells in a multiwall plate (1 mL per well). The multiwall plate was placed in a laminar flow hood until the concentrated collagen-hyaluronic acid composition comprising the active agent was dry.

To prepare Latanoprost collagen films, 500 μg of a Latanoprost active agent was mixed with 4 mL of crosslinked HA-collagen compositions as prepared above. The mixture was centrifuged at 3500 rpm for 10 minutes if air bubbles were observed. The mixture was then poured into 35 mm wells in a multiwall plate (1 mL per well). The multiwall plate was placed in a laminar flow hood until the concentrated collagen plus Latanoprost was dry.

### UV Irradiation to Polymerize Collagen Films

The dried collagen-hyaluronic acid film was placed in a controlled atmosphere chamber containing an ultraviolet lamp emitting 254 nm of irradiation. Prior to activating the ultraviolet lamp, the chamber was flushed with nitrogen gas. After 5 minutes of flushing, the exit port was closed, the ultraviolet lamp activated and the collagen composition exposed to 254 nm ultraviolet irradiation for 18 minutes while continuing to flush the chamber to maintain chamber expansion.

### Evaluation of Active Agent Release In Vitro

Four round pieces of 35 mm diameter polymerized film were cut into 4 sections and placed in 1.5 mL vials containing 1 mL of 0.01 M phosphate buffer at a pH of 7.4. The 16 vials were placed in a controlled temperature chamber at 37° C. Five aliquots of 100 μL were removed from 2 vials each at 1 hour, 3 days, 7 days, 2 weeks, 1 month, 2 months, and 3 months. Two vials were reserved for additional time periods if release was still noted at 3 months. Four vials of buffer only were used for controls.

### Analysis of Active Agent

Active agent concentration (e.g., Latanoprost) was measured by HPLC at Millennium Research Laboratories. Detection at nanogram levels was validated using active agent (e.g., Latanoprost) standards and concentration curves. Results showed an initial bolus release at 1 day followed by sustained, zero order release through 3 months.

### Degradation of Collagen Films

Trypsin Digestion: Degradation of collagen films was evaluated by incubating films without Latanoprost in a 0.02% trypsin solution containing 1M Trizma buffer, pH 8.0 containing sodium chloride, calcium chloride and magnesium chloride. After 24 hours of incubation at 37° C., vials were centrifuged to recover the supernatant and analyzed for hydroxyproline using standard assays. The degradation profile was determined by calculating the hydroxyproline present in the incubation supernatant, which was indicative of collagen degraded as a result of trypsin digestion. The results showed negligible degradation of collagen film when incubated in trypsin.

Collagenase Digestion: Degradation of collagen films was evaluated by incubating films without Latanoprost in bacterial collagenase in a sodium phosphate buffer at a pH of 7.4. After 24 hours of incubation at 37° C., vials were centrifuged to recover the supernatant and analyzed for hydroxyproline using standard assays. The degradation profile was determined by calculating the hydroxyproline present in the incubation supernatant, which was indicative of collagen degraded as a result of collagenase digestion. The results showed minimal degradation of collagen film when incubated in collagenase.

### Therapeutic Potential of Drug Released from Collagen-Hyaluronic Acid Films

The results of active agent release experiments and collagen digestion experiments demonstrated the potential of providing a durable implant for sustained release of active agent for effective treatment of glaucoma.

### Testing of Release Profile

A Latanoprost-containing collagen film as described in Example 1 was evaluated for the release of active agent over time. The results are set forth in FIG. 5.

### Example 2

### Preparation of PLL Grafted to HA

1 gram of LifeCore hyaluronic acid (1 MM molecular weight) and 100 milligrams of PLL were dissolved in 150 ml of sodium borate buffer (0.1M, pH 8.5) containing 1M NaCl. A sodium borohydride solution (NaBH₃CN; in borate buffer) was added directly to the mixture to a concentration of 25 mM. The mixture was stirred and incubated at room temperature for 48 hours. The reactant was then dialyzed against 0.5M NaCl for 3 days. The molecular weight of the hyaluronic acid component was approximately 1.5×10⁶. PLL was purchased from Sigma Aldrich (Poly-L-lysine hydrobromide, molecular weight 4,000-15,000). In some embodiments, the UV-crosslinked collagen construct is further treated by incubating it in a solution, for example a saline solution. In some embodiments, the solution treatment is for about 1 day, 2 days, 3 days, 4 days, 5 days, 6 days, 7, days, about 1 week, about 2 weeks, or about 3 weeks. In some embodiments the solution treatment results in collagen construct that releases drug with zero order kinetics or with near zero order kinetics. In still other embodiments, the solution treatment results in collagen-based construct that releases drug with an initial bolus until about day 1, day 2, or day 3, followed by controlled release for at least about 3, 4, 5, 6, 7, 8, 9, 10, 11, or 12 months. In still further embodiments, the controlled release is for at least 6 months, but not for more than about 7, 8, 9, 10, 11, or 12 months.

### Preparation of Derivatized Collagen

Two hundred milliliters of 3 mg/mL purified, soluble collagen (Porcogen, Lot #531131080) was filtered through 0.45 um and 0.2 um cartridge filters. The filtered collagen was place in a 500 ml beaker and adjusted to a pH 9.0 using 10N and 1N NaOH. After stirring for 5 minutes at room temperature, pulverized glutaric anhydride powder (Sigma, >95%) was slowly added to the stirring collagen solution at a concentration equal to 10% of the collagen (60 mg). The pH of the collagen solution was maintained at pH 9.0 by addition of drops of 10N NaOH. The glutaric anhydride reaction continued for 15 minutes, at which point drops of 6N HCl and 1 HCl were added to reduce the pH top approximately 4.5 to precipitate the derivatized collagen. The derivatized collagen was then placed in 50 mL centrifuge tubes and centrifuged at 3500-5000 rpm to precipitate the derivatized collagen. The recovered precipitate was then solubilized by adjusting the pH to 7.2 by adding drops of 10N NaOH and 1N NaOH. The pH was monitored as the NaOH was mixed with the derivatized collagen pellet. The neutralized, clear and transparent collagen gel was then placed in 50 mL centrifuge tubes and centrifuged to remove air bubbles.

### Polymerization of HA-PLL and Derivatized Collagen Using Bifunctional Acylating Agents-Diglutaryl Chloride or EDTA Dianhydride

HA-PLL solutions were prepared at about 1% HA. Derivatized collagen was prepared at a concentration of 3% collagen. The HA-PLL and derivatized collagen were adjusted to a pH of 9.5 and the mixture stirred for 15 minutes. A transparent, viscous solution formed. The mixture was crosslinked with 1% diglutaryl chloride solution. The solution was dialyzed against 0.04M sodium phosphate buffer containing 0.9% NaCl. The solution was then isolated from the dialysis tubing and stored at 2-8° C.

A second crosslinked HA-PLL and derivatized collagen solution were crosslinked by reacting the HA-PLL solution with 10% ethylenediaminetetraacetic dianhydride (Sigma Aldrich Chemical Company, 97%). Resultant HA-PLL and derivatized collagen solutions were transparent and very thick (gel-like). The product was dialyzed against 0.04M sodium phosphate buffer containing 0.9% NaCl. The product was then isolated from the dialysis tubing and stored at 2-8° C.

### Visual Examination of Diglutaryl Chloride Polymerized HA-PLL Derivatized Collagen Compositions (HA-PLL-Collagen)

A droplet of free acid Fluorescein dissolved in 0.1M Sodium Phosphate buffer, pH 7.2 was added to 2 mL of HA-PLL-Collagen polymerized using diglutaryl chloride. Fifty microliters (50 uL) of the fluorescein stained HA-PLL-Collagen/diglutaryl chloride gel was injected through a 27 G needle into 2 mL of 0.1M sodium phosphate buffer to evaluate the appearance of the compositions. The droplet formed a continuous thread upon injection and appeared to maintain physical structure for more than days.

### Example 3

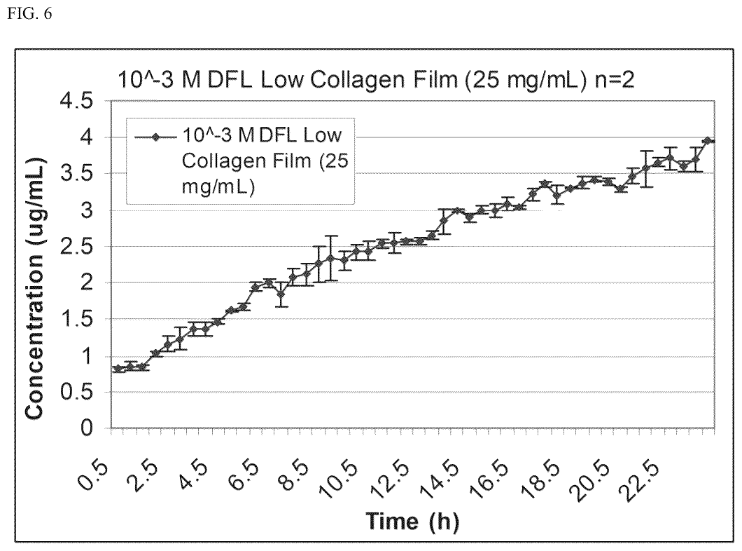

A further experiment was conducted to test the transscleral diffusion of an active agent (0.01 M dexamethasone-fluorescein (DFL) over time from a low concentration collagen (25 mg/mL) film as described above. The results are reported in FIG. 6.

### Example 4

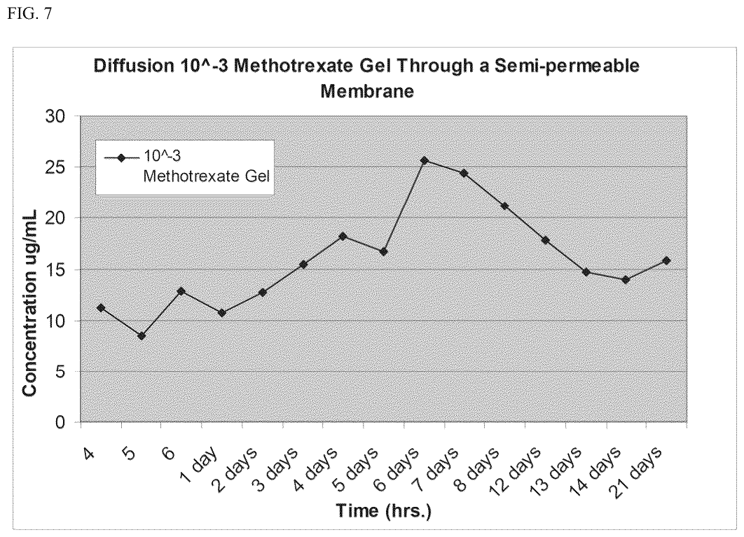

A further experiment was conducted similar to that of Example 3. In this experiment, the transscleral diffusion of an active agent (0.01 M methotrexate gel) over time was evaluated for a low concentration collagen film as described above. The results are reported in FIG. 7.

The results of both Example 3 and 4 clearly demonstrated the effectiveness of ocular drug delivery from unique collagen-based films.

### Example 5

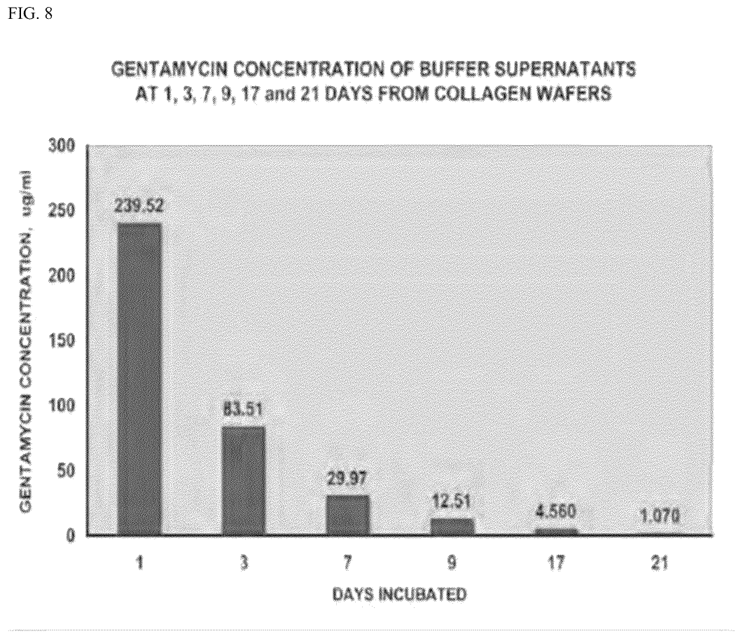

Another experiment was conducted to test the delivery of an active agent from a collagen film described above, exposed to limited UV curing. In this experiment, gentamycin was the active agent and the concentration of buffer supernatants from the film were evaluated over time. The results are reported in FIG. 8.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above products and methods without departing from the scope of the invention, it is intended that all matter contained in the above description and shown in the accompanying drawings shall be interpreted as illustrative and not in a limiting sense.

We claim:

1. A collagen-hyaluronic acid based film or membrane delivery system comprising:
one or more layers comprising crosslinked, chemically derivatized collagen-hyaluronic acid;
wherein the crosslinked, chemically derivatized collagen-hyaluronic acid comprises chemically derivatized collagen crosslinked with a hyaluronic acid.

2. The film or membrane delivery system of claim 1, comprising:
two or more layers of crosslinked, chemically derivatized collagen-hyaluronic acid;
wherein at least one layer is a rate controlling layer having multiple surfaces composed of derivatized collagen on at least one surface of the multiple surfaces.

3. The film or membrane delivery system of claim 1, wherein the collagen is derivatized with an acylating agent to alter the ionic charge and charge density.

4. The film or membrane delivery system of claim 3, wherein the acylating agent is selected from the group consisting of anhydrides including maleic anhydride, succinic anhydride, glutaric anhydride, citractonic anhydride, methyl succinic anhydride, itaconic anhydride, methyl glutaric anhydride, dimethyl glutaric anhydride, phthalic anhydride; acid chlorides including oxalyl chloride, malonyl chloride; sulfonyl chlorides including chlorosulfonylacetyl chloride, chlorosulfonylbenzoic acid, 4-chloro-3-(chlorosulfonyl)-5-nitrobenzoic acid, and 3-(chlorosulfonyl)-P-anisic acid; sulfonic acids including 3-sulfobenzoic acid; and combinations thereof.

5. The film or membrane delivery system of claim 1, wherein collagen is altered to increase the net positive charge by reacting the collagen solution with an acylating agent comprising 4,6-diamino-2-methylthiopyrimidine-5-sulfonic acid.

6. The film or membrane delivery system of claim 1, wherein the film or membrane further comprises extended therapeutic concentrations of an active agent and the film or membrane is exposed to ultraviolet irradiation in a nitrogen atmosphere for time periods ranging from about 2 minutes to about 14 minutes, preferably about 5 minutes to about 12 minutes.

7. The film or membrane delivery system of claim 1, further comprising extended therapeutic concentrations of drugs selected from the group consisting of tetracycline, chlortetracycline, bacitracin, neomycin, polymyxin, gramicidin, oxytetracycline, chloramphenicol, gentamycin, penicillin, kanamycin, amikacin, sisomicin, tobramycin, garamycin, ciprofloxacin, norfloxacin and erythromycin; antibacterials such as sulfonamides, sulfacetamide, sulfamethizole and sulfisoxazole; antivirals, including idoxuridine; and other antibacterial agents such as nitrofurazone and sodium propionate; anti-allergenics such as antazoline, methapyriline, chlorpheniramine, pyrilamine and prophenpyridamine; anti-inflammatories such as cortisone, hydrocortisone, hydrocortisone acetate, dexamethasone, dexamethasone 21-phosphate, fluocinolone, medrysone, prednisolone, methylprednisolone, prednisolone 21-phosphate, prednisolone acetate, fluorometholone, betamethasone, fluocortolone, indomethacin and triamcinolone; decongestants such as phenylephrine, naphazoline and tetrahydrozoline; miotics and anti-cholinesterase's such as pilocarpine, eserine salicylate, carbachol, di-isopropyl fluorophosphate, phospholine iodide, echothiophate, physostigmine and demecarium bromide; mydriatics such as atropine sulfate, cyclopentolate, homatropine, scopolamine, tropicamide, eucatropine, and hydroxyamphetamine; sympathomimetics such as epinephrine and immunosuppressants such as cyclosporin and azathioprine combined with acylated collagen at neutral pH; and combinations thereof.

8. A method of using the film or membrane delivery system of claim 1 for treatment of glaucoma, comprising placing the film or membrane intraocularly in the anterior chamber, posterior chamber, or vitreous or placing the film or membrane in the lacrimal canaliculus after dilating the punctum.

9. The film or membrane delivery system of claim 1, wherein the films or membranes further comprise an extended therapeutic concentration of an active agent, wherein the concentration is at least about 150 μg, at least about 175 μg, at least about 200 μg, at least about 225 μg, at least about 250 μg, at least about 275 μg, at least about 300 μg, at least about 325 μg at least about 350 μg, at least about 400 μg, at least about 450 μg, or at least about 500 μg per film or membrane to provide an effective sustained, zero-order delivery dosage for from about 1 week to about 6 months, from about 2 weeks to about 6 months, from about 2 weeks to about 5 months, from about 2 weeks to about 4 months, from about 2 weeks to about 3 months, from about 2 weeks to about 2 months, or from about 2 weeks to about 1 month.

10. A method of preparing a derivatized collagen-hyaluronic acid based film or membrane delivery system, comprising:
preparing a derivatized collagen and dissolving the derivatized collagen in a buffer;
mixing the derivatized collagen in a buffer with a hyaluronic acid to form a mixture;
adding an active agent to the mixture to form a solution;
casting the solution into a layer and allowing the layer to dry or partially dry; and
exposing the dry or partially dry layer to ultraviolet irradiation in a nitrogen atmosphere to form the derivatized collagen-hyaluronic acid based film or membrane delivery system.

11. The method of claim 10, wherein the hyaluronic acid is a crosslinked hyaluronic acid prepared by crosslinking hyaluronic acid and a polymer.

12. The method of claim 11, wherein Poly-L-lysine hydrobromide (PLL) is crosslinked with hyaluronic acid to form the crosslinked hyaluronic acid HA-PLL.

13. The method of claim 10, wherein mixing the derivatized collagen in a buffer with a hyaluronic acid comprises crosslinking the derivatized collagen and hyaluronic acid with an acylating agent.

14. A method for preparing a collagen-hyaluronic acid based gel, comprising:
crosslinking Poly-L-lysine hydrobromide (PLL) and hyaluronic acid to form a HA-PLL gel;
derivatizing soluble collagen to form a derivatized collagen gel;
mixing the HA-PLL gel with the derivatized collagen gel and adjusting the pH to 9.5;
adding a bifunctional acylation agent to the mixture to produce a gel; and
adding 1N NaOH to the gel to adjust the pH to from about 6.8 to about 7.4.

15. The method of claim 14 further comprising exposing the collagen-hyaluronic acid based gel to ultraviolet irradiation.

16. The method of claim 14 wherein the mixture of HA-PLL gel and derivatized collagen gel has a hyaluronic acid concentration from about 1 to about 3 wt % and a collagen concentration from about 1 to about 5 wt %.

17. The method of claim 14, further comprising mixing an active agent with the HA-PLL gel and derivatized collagen gel.

18. The method of claim 14, further comprising placing the gel into a mold and exposing the gel to ultraviolet irradiation in a nitrogen atmosphere to form a film.

19. The method of claim 18, wherein the film is deposited manually in a selected ocular location for sustained drug delivery for a desired period of time, wherein depositing manually comprises the use of forceps or a syringe type delivery system.

20. The method of claim 19, wherein the procedure is reversible and the construct is removed using the forceps or syringe type delivery system.
